(19) United States
(12) Patent Application Publication
(10) Pub. No.: US 2013/0149728 A1
(43) Pub. Date: Jun. 13, 2013

(54) Oral Fluid Sample Collection Device With Indicator and Method

(71) Applicant: Cytosignet, Inc., North Andover, MA (US)

(72) Inventors: Nathan L. Smith, North Andover, MA (US); Debra Owen Stabu, Franklin, MA (US)

(73) Assignee: CYTOSIGNET, INC., North Andover, MA (US)

(21) Appl. No.: 13/707,760

(22) Filed: Dec. 7, 2012

Related U.S. Application Data
(60) Provisional application No. 61/568,786, filed on Dec. 9, 2011.

(51) Int. Cl. C12Q 1/54

(52) U.S. Cl. CPC C12Q 1/54
USPC 435/14; 435/34; 436/66; 436/164; 422/430

(57) ABSTRACT

Provided are methods and devices used for determining the presence or absence of a contaminant in the sample of oral fluid. The method involves contacting a substrate having a detectable reporter with a sample of oral fluid and, based on determining a signal from the detectable reporter determining the presence or absence of a contaminant in the sample of oral fluid. The invention is useful for detecting lactose, milk, colostrum, blood, hemoglobin, whole cells, and combinations thereof. The method can be used for detecting oral contaminants in oral fluid samples obtained from any mammal, including neonatal ungulates.

### CROSS-REFERENCE TO RELATED APPLICATIONS

This application claims priority to U.S. provisional patent application No. 61/568,786, filed Dec. 9, 2011, the disclosure of which is incorporated herein by reference.

### FIELD OF THE INVENTION

This invention relates generally to medical devices and more specifically to medical devices for determining the presence or absence of contaminants in oral fluid samples.

### BACKGROUND OF THE INVENTION

Oral fluid samples obtained for various biochemical analyses are potentially subject to contamination by ingestion (feeding) activity and/or blood.

### BRIEF SUMMARY OF THE INVENTION

The present invention provides methods, devices and kits useful for determining the presence or absence of a contaminant in the sample of oral fluid. The method generally involves contacting a substrate having a detectable reporter with a sample of oral fluid and, based on determining a signal from the detectable reporter determining the presence or absence of a contaminant in the sample of oral fluid. The invention is useful for detecting a variety of contaminants that could be present in an oral fluid sample obtained from any mammal. The contaminants include but are not limited to lactose, milk, colostrum, blood, hemoglobin, whole cells, and combinations thereof. In one embodiment, the method and kits of the invention including are suitable for detecting contaminants in oral fluid samples obtained from neonatal ungulates.

### BRIEF DESCRIPTION OF THE FIGURES

FIG. 1. Glucose color chart from the labeling experiments performed.

FIG. 2. The top strip, the pad was the normal glucose reagent pad. In the bottom strip, lactase was added. Both strips were used to test colostrum from a llama. The glucose pad showed no reaction (negative for glucose) and the modified glucose pad showed a positive reaction for the glucose released from lactose by the action of the lactase enzyme.

FIG. 3. Oral fluid samples from neonatal calf (“Bull 30”), colostrum replacer (dried bovine colostrum reconstituted in water and diluted “Col Rep 1:10” ibid.), and bovine colostrum (“Colostrum” ibid.) were applied to the modified glucose test pads of urine test strips (modified by addition of 9 I.U. of lactase). Two samples of oral fluid, collected at the time of birth “0 H”) and 2 days after birth (“48 H”) were negative for glucose whereas colostrum, either as replacer or as collected, were positive for glucose and therefore lactose.

FIG. 4. Oral fluid samples were collected from one alpaca cria at two times within the first day of life. They were tested with modified glucose pads. This cria was allowed to nurse from the dam at will before Sample #2 (bottom pad in figure) was collected. The figure gives an indication sample #2 (lower strip) was contaminated with colostrum whereas sample #1 (top) was not.

FIG. 5. Oral fluid samples collected from a foal were similarly tested for contamination by colostrum with modified and unmodified glucose reagent pads with the same results as were obtained with cria samples as shown. Foal saliva sample #76 was collected soon after feeding so contamination by colostrum was expected.

FIG. 6. Foal oral fluid sample #72 was not contaminated with colostrum did not give a color change with modified glucose.

FIG. 7. Human oral fluid samples were collected before and after the test subject drank milk. The samples were tested with glucose reagent pads modified as described herein. The result, shown in the figure, shows that the oral fluid sample contaminated with milk developed a color change indicating a positive reaction for glucose (bottom strip) which did not occur with the sample collected before ingesting milk (top strip).

FIG. 8. A oral fluid sample (foal #72 shown in FIG. 6) and bovine colostrum were tested with all the test pads except glucose on urine test strips (URS-10 from Teco Diagnostics). The following figure shows the test name on the left and the negative (no reaction, i.e. none detected) color from the comparison chart for the test. Two test strips—oral fluid on the left and colostrum on the right are positioned to allow direct comparison with the color chart. Significant differences between the two samples are clearly visible in three determinations: protein, blood (hemoglobin), and specific gravity.

### DETAILED DESCRIPTION OF THE INVENTION

The present invention provides novel methods using a variety of devices to determine the presence or absence of contaminants in oral fluid samples.

Briefly, the present invention teaches oral fluid collection with an indication of contamination at or near the time of collection. In various embodiments, the invention includes incorporating into a collection device an absorbent material impregnated with indicator reagents (i.e., detectable reporters) to detect suspected potential alteration of the composition or properties of the sample. For example, the oral fluid may be contaminated with colostrum which could potentially affect analysis of the sample for total protein, specific proteins such as immunoglobulins or antibody activity, carbohydrates, lipids, among other biochemical compounds. Additionally, for example, ingestion of water could result in erroneously low levels of all constituents of oral fluid due to the dilution effect of water. Contamination of oral fluid by blood, as another example, would introduce cells into the sample in addition to adding the constituents of blood serum.

In one embodiment, the invention provides a collection device comprising a detectable reporter suitable for detecting a contaminant in an oral fluid, and further comprising an oral fluid. Also provided are kits. The kits comprise a collection device of the invention and components for collecting oral fluid.

Teachings of the present invention provide for a visible or otherwise readable indication that the sample of oral fluid could contain certain undesirable components.

The following descriptions are presented to demonstrate the invention and its versatility and are not intended to be limiting.

In one embodiment, the invention provides for determining the presence or absence of colostrum in a sample of oral fluid. For instance, colostrum could readily and rapidly be indicated by detecting, for example, the presence of lactose in the normally lactose free oral fluid. Because lactose is similarly found in milk, the same indicator could be used to identify oral fluid contaminated by milk, or identify oral fluid that is not contaminated by milk, or to identify colostrum. In another embodiment, contamination of an oral fluid sample by blood would be indicated by the unexpectedly increased presence of hemoglobin in the sample. Contamination by either blood or colostrum would also be expected to increase the specific gravity of the oral fluid sample and such an increase would indicate potential alteration of the integrity of the sample. Additionally, colostrum, milk, or blood contamination would increase the protein content of oral fluid and, therefore, a protein measurement would indicate contamination. Recent ingestion of water could dilute the oral fluid which could be indicated by abnormally low specific gravity.

The descriptive examples in the immediately foregoing paragraph were selected because very simple and rapid colorimetric tests currently exist for use with urine samples. The reagents can be put to novel use to help assure the integrity of oral fluid or saliva samples. Use of these single-step, point-of-sample-collection, and single-use chemistry reagents would be reliable and cost effective. For example, blood contamination in an oral fluid sample could be indicated using the hemoglobin pad of urine strips. Also using standard urine strip chemistries, blood contamination might also be indicated by higher than expected protein or glucose, both of which would enter the sample with the blood contamination and not oral fluid. Dilution of oral fluid by water could be indicated by specific gravity measurements using, for example, urine test strip chemistry. Colostrum contamination would be expected to increase protein concentration in, for example, neonatal mammal oral fluid. By combining standard urine test strip test for glucose with lactase would allow for detecting mammary fluid contamination of oral fluid samples due to the presence of lactose.

The present invention is not intended to be solely dependent upon urine test strip chemistry to provide time-of-collection indication of a contaminated oral fluid sample. Any rapid test that would identify contamination of oral fluid samples at, for example, the time and site of collection could be employed.

Practice of the present invention would be advantageously employed in combination with, for example, measurement of immunoglobulins in the oral fluid of neonatal ungulates as disclosed in U.S. patent publication no. 2010/0111934, the disclosure of which is incorporated herein by reference, in addition to other instances of oral fluid collection.

The following example is presented to illustrate the present invention. It is not intended to limiting in any manner.

### EXAMPLE 1

This Example demonstrates an embodiment of the invention using oral fluid samples collected from calves, foals, humans, and cria for colostrum and milk. Urine test strips (Chemstrip 10 manufactured by Roche Diagnostics unless otherwise noted) were used. These commercially available in-vitro diagnostic devices have ten individual pads, each of which contains chemical reagents to perform a specific test. For the individual chemistry testing described herein, the glucose pad was used. To meet the requirements of the present invention, the glucose pads were used as provided or modified by adding the lactose hydrolyzing enzyme “Lactase”, more specifically commercially available E. coli (β-galactosidase (purchased from ABD Serotec), Approximately 10 I.U. of lactase was added prior to applying the sample to be tested.

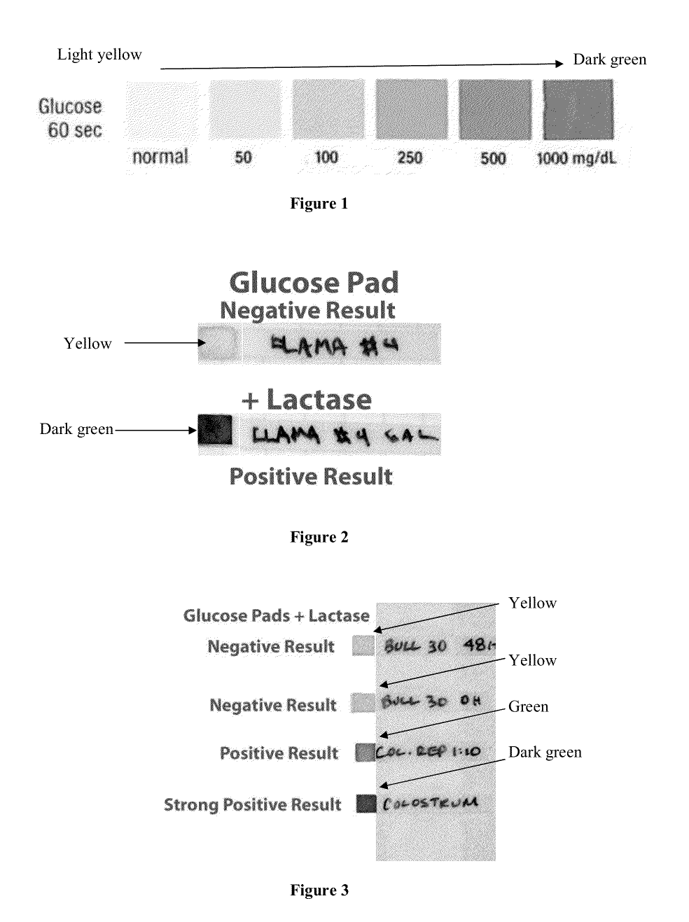

The figures presented herein were edited to show only the glucose pad. In all cases the results were read visually as directed by the instructions provided by the manufacturer. After reading, interpreting, and recording the result photographs (shown in FIGS. 2-8) were taken. These photographs (shown in FIGS. 2-8) are indicative of the test results but are not always as clear as by reading by eye. In the results presented herein, the visual interpretation is given along with the photographic representation of the test (shown in FIGS. 2-8). With the glucose pad and the modified glucose pad, prepared by adding lactase as described, the yellow color indicates no reaction, i.e., negative for detectable glucose. The green/blue indicates positive detection of glucose and the intensity of the darker color is proportional to the amount of glucose present (FIG. 1). Using this correlation between visible color and amount of glucose detected, the manufacturer permits the user to interpret the test result quantitatively. The glucose color chart from the labeling is shown in FIG. 1.

For the purpose of these examples, the presence of color development in the modified glucose pads represents a glucose detection either from glucose itself or that hydrolyzed from lactose.

Lactose, known as “milk sugar”, is present in the mammary gland products colostrum and milk. Lactose, a disaccharide of galactose and glucose does not react with the urine strip chemistry for glucose as shown in FIG. 2. In order to test for lactose, the lactose hydrolyzing enzyme lactase (β-galactosidase) was added to the glucose pad to produce glucose from lactose and, if lactose were present, provide a positive reaction with the thus modified glucose pad. In FIG. 2, the top strip, the pad was the normal glucose reagent pad. In the bottom strip, lactase was added. Both strips were used to test colostrum from a llama. The glucose pad showed no reaction (negative for glucose) and the modified glucose pad showed a positive reaction for the glucose released from lactose by the action of the lactase enzyme (FIG. 2).

Oral fluid samples from neonatal calf (“Bull 30” in FIG. 3), colostrum replacer (dried bovine colostrum reconstituted in water and diluted “Col Rep 1:10” ibid.), and bovine colostrum (“Colostrum” ibid.) were applied to the modified glucose test pads of urine test strips (modified by addition of 9 I.U. of lactase). Two samples of oral fluid, collected at the time of birth “0 H”) and 2 days after birth (“48 H”) were negative for glucose whereas colostrum, either as replacer or as collected, were positive for glucose and therefore lactose (FIG. 3). These results show that oral fluid contains neither glucose nor lactose in sufficiently high concentration to give positive reaction with the glucose or modified glucose reagent pads.

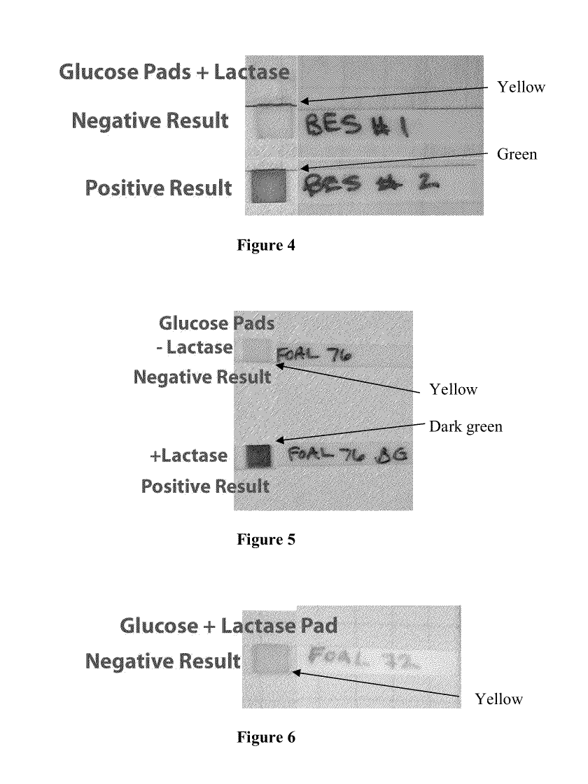

Oral fluid samples were collected from one alpaca cria at two times within the first day of life. They were tested with modified glucose pads. This cria was allowed to nurse from the dam at will before Sample #2 (bottom pad in FIG. 4) was collected. FIG. 4 gives an indication sample #2 (lower strip) was contaminated with colostrum whereas sample #2 (top) was not.

Oral fluid samples collected from a foal were similarly tested for contamination by colostrum with modified and unmodified glucose reagent pads with the same results as were obtained with cria samples as shown. Foal saliva sample #76 was collected soon after feeding so contamination by colostrum was expected (FIG. 5).

Foal oral fluid sample #72 was not contaminated with colostrum did not give a color change with modified glucose (FIG. 6).

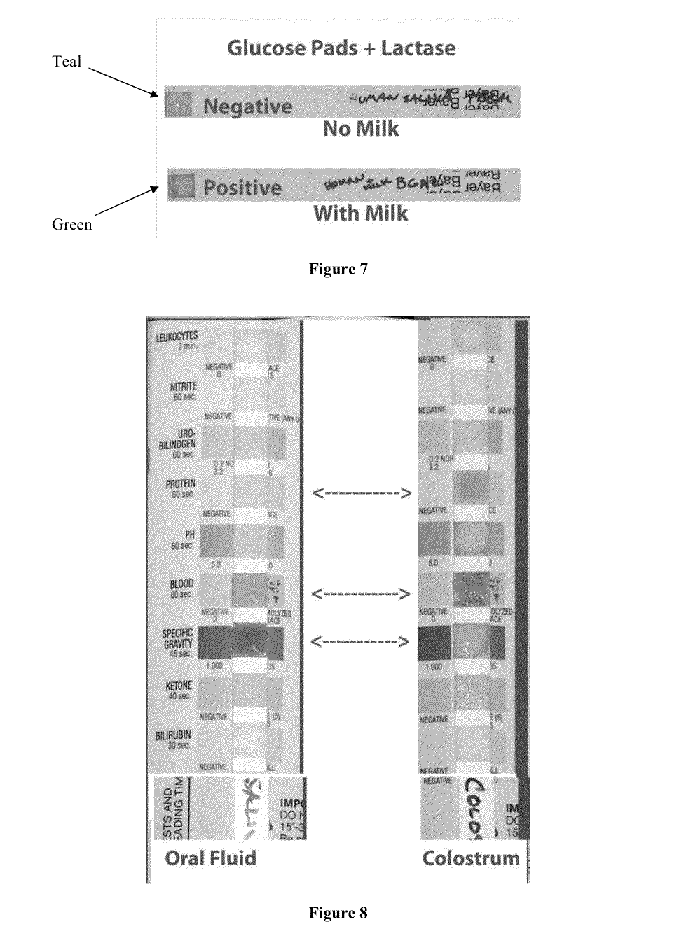

Human oral fluid samples were collected before and after the test subject drank milk. The samples were tested with glucose reagent pads modified as described herein. The result, shown in FIG. 7, shows that the oral fluid sample contaminated with milk developed a color change indicating a positive reaction for glucose (bottom strip) which did not occur with the sample collected before ingesting milk (top strip). The results are shown in FIG. 7.

An oral fluid sample (foal #72 shown in an earlier example) and bovine colostrum were tested with all the test pads except glucose on urine test strips (URS-10 from Teco Diagnostics). FIG. 8 shows the test name on the left and the negative (no reaction, i.e. none detected) color from the comparison chart for the test. Two test strips—oral fluid on the left and colostrum on the right are positioned to allow direct comparison with the color chart (FIG. 8). Significant differences between the two samples are clearly visible in three determinations: protein, blood (hemoglobin), and specific gravity. These results indicate that these pads, in addition to glucose, could be used for identifying contamination of oral fluid samples.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1. A method for analysis of oral fluid comprising contacting a substrate having a detectable reporter with a sample of oral fluid and, based on determining a signal from the detectable reporter determining the presence or absence of a contaminant in the sample of oral fluid.

2. The method of claim 1, wherein the contaminant is selected from the group consisting of lactose, milk, colostrum, blood, hemoglobin, whole cells, and combinations thereof.

3. The method of claim 2, where the contaminant is lactose.

4. The method of claim 3, wherein determining presence of lactose is indicative of the presence of colostrum in the sample of oral fluid, and wherein determining absence of lactose is indicative of the absence of colostrum in the sample of oral fluid.

5. The method of claim 1, wherein the sample of oral fluid is obtained from a mammal.

6. The method of claim 5, wherein the mammal is a neonatal ungulate.

7. The method of claim 6, wherein determining an absence of a contaminant in the sample of oral fluid is indicative that the sample of oral fluid is suitable for determining whether or not the neonatal ungulate has failure of passive transfer of immunity.

8. A composition comprising a substrate having a detectable reporter suitable for detecting contaminants of oral fluid, and further comprising an oral fluid.

9. A kit for determining contamination of oral fluid, the kit comprising a substrate having a detectable reporter suitable for detecting contaminants of oral fluid and components for collection of an oral fluid.
